(19) United States
(12) Patent Application Publication
(10) Pub. No.: US 2007/0042363 A1
(43) Pub. Date: Feb. 22, 2007

(54) Novel polypeptide and nucleic acid encoding the same

(75) Inventors: Yoshihide Hayashizaki, Ibaraki (JP); Harukazu Suzuki, Kanagawa (JP); Mutsumi Kanamori, Kanagawa (JP); Matthias Harbers, Tokyo (JP)

Correspondence Address: BIRCH STEWART KOLASCH & BIRCH, PO BOX 747, FALLS CHURCH, VA, 22040-0747, US

(73) Assignees: The Institute of Physical and Chemical, Wako-shi, Saitama (JP); Kabushiki Kaisha Dnaform, Tokyo (JP)

(21) Appl. No.: 10/509,727

(22) PCT Filed: Jan. 22, 2003
(86) PCT No.: PCT/JP03/00522

§ 371 Date: Aug. 16, 2005

(30) Foreign Application Priority Data
Apr. 2, 2002 (JP) 2002-100431

(51) Int. Cl. C12Q 1/68; C07H 21/04; C12P 21/06; C07K 14/705

(52) U.S. Cl. 435/6; 530/350; 435/69.1; 435/320.1; 435/325; 536/23.5

(57) ABSTRACT

A novel protein which binds with tumor necrosis factor receptor associated factor 2, TRAF2, as well as a nucleic acid coding for the protein is disclosed. From a mouse cDNA library, a cDNA coding for a novel protein which binds with TRAF2 was discovered by mammalian two-hybrid assay, the cDNA was sequenced, the protein encoded by the cDNA was produced, and the fact that the protein binds with TRAF2 was experimentally confirmed.

### TECHNICAL FIELD

The present invention relates to a novel polypeptide which binds with tumor necrosis factor receptor-associated factor 2 (TRAF2) and a nucleic acid coding for the same.

### BACKGROUND ART

Tumor necrosis factor receptor-associated factors (TRAFs) are known as adaptor proteins that link the TNF receptor family to signaling pathways. The TRAF gene family consists of six members denoted as TRAF1 to TRAF6, and is characterized by the conserved TRAF domain at carboxyl-terminal of each protein (1-5). In the TRAF family, TRAF2 is involved in TNF-mediated signaling pathway.

When TNF receptor 1 (TNFR1) is stimulated by TNF, TNFR1 recruits TRAF2 indirectly via the amino-terminal domain of TRADD (TNFR-associated death domain protein) (6, 7). The activated TRAF2 recruits several proteins including RIP (8, 9) and ASK1 (10), which mediate the activation of NF-κB and AP-1, resulting in induction of many genes in cellular and immune functions (11, 12).

Besides the TRAF2 interaction, TRADD also interacts with FADD (13), an adaptor molecule with death domain. FADD recruits and activates caspase-8, an initiation protease of death protease cascade, resulting in apoptosis (14). Therefore, TRAF2 is a key molecule involved in TNF-mediated cell survival where a variety of proteins regulate the signaling pathway by the interaction with TRAF2.

Therefore, discovery of a novel TRAF2 binding protein and characterization thereof are important for the understanding of physiological and pathological processes involved in the TNF-mediated signaling pathway. Further, such a protein may have a use for the diagnosis and therapy of diseases in which the TNF-mediated signaling pathway is involved.

Accordingly, an object of the present invention is to provide a novel protein which binds with TRAF2 and a nucleic acid coding for the same.

The present inventors intensively studied to discover a cDNA coding for a novel protein which binds with TRAF2 from a mouse cDNA library by the mammalian two-hybrid assay, and experimentally confirmed that the protein encoded by the cDNA binds with TRAF2, thereby completing the present invention.

That is, the present invention provides a polypeptide comprising a region having the amino acid sequence from 1st to 162nd amino acid in the amino acid sequence shown in SEQ ID NO:1 in SEQUENCE LISTING or a region having the same amino acid sequence as said amino acid sequence from 1st to 162nd amino acid in the amino acid sequence shown in SEQ ID NO:1 except that 1 to 40 amino acid residue(s) is(are) substituted, deleted and/or inserted, which polypeptide has a binding ability to TRAF2. The present invention also provides a polypeptide comprising a region having the amino acid sequence from 1st to 162nd amino acid in the amino acid sequence shown in SEQ ID NO:3 in SEQUENCE LISTING or a region having the same amino acid sequence as said amino acid sequence from 1st to 162nd amino acid in the amino acid sequence shown in SEQ ID NO:3 except that 1 to 40 amino acid residue(s) is(are) substituted, deleted and/or inserted, which polypeptide has a binding ability to TRAF2. The present invention further provides a nucleic acid coding for the above-described polypeptide according to the present invention. The present invention further provides an expression vector containing the nucleic acid according to the present invention, which can express the nucleic acid in a host cell. The present invention still further provides a nucleic acid which hybridizes with the nucleic acid according to the present invention, and which may be used for the detection of the nucleic acid according to the present invention. The present invention still further provides a method for measuring the nucleic acid according to the present invention using the nucleic acid as a probe or primer.

By the present invention, a novel polypeptide binding with TRAF2 and a nucleic acid coding for the same were first provided. Since the polypeptide according to the present invention binds with TRAF2, it is important for the studies of TRAF2-mediated signaling. Further, since the polypeptide of the present invention activates NF-κβ signal, which is suggested to be a TNA-mediated signaling, the NF-κβ signal may be attenuated by inhibiting the function of thereof. Thus, the polypeptide is useful as a target for the development of therapeutic drugs against inflammation, rheumatism or the like. Further, since NF-κβ signal is involved in the activation of osteoclasts, it is useful as a target for the development of therapeutic drugs against osteoporosis.

### BRIEF DESCRIPTION OF THE DRAWINGS

FIG. 1A shows the result of mammalian two-hybrid assay; and FIG. 1B shows amino acid sequences of mouse and human T2BPs in comparison.

FIG. 2A shows schematic representation of TRAF2 and the deletion mutants and their interaction with wild-type T2BP; and FIG. 2B shows schematic representation of T2BP and the deletion mutants and their interaction with wild-type TRAF2.

FIG. 3 shows the result of the co-immunoprecipitation carried out in Example of the present invention, indicating that T2BP binds with TRAF2.

FIG. 4 shows the results of Northern blot analysis carried out in Example of the present invention, indicating the expression of T2BP in various tissues.

FIG. 5 shows the relationship between the amount of T2BP and the relative luciferase activity, indicating the activation of NF-κB and AP-1 in 293 cells transfected with T2BP, which was carried out in Example of the present invention.

### BEST MODE FOR CARRYING OUT THE INVENTION

By the method detailed in Example below, a cDNA coding for a novel polypeptide which binds with TRAF2 was discovered from a cDNA library originated from thymus of a 3-day old mouse by the mammalian two-hybrid assay. The nucleotide sequence is shown in SEQ ID NO:4 together with the deduced amino acid sequence encoded thereby. SEQ ID NO:3 shows the amino acid sequence alone shown in SEQ ID NO:4. This polypeptide was named “T2BP” (TRAF2-binding protein). By homology search by BLAST, a cDNA having a high homology with the mouse T2BP cDNA was discovered from human genes whose functions are unknown. The nucleotide sequence of this human cDNA is shown in SEQ ID NO:2 together with the deduced amino acid sequence encoded thereby. SEQ ID NO:1 shows the amino acid sequence alone shown in SEQ ID NO:2. Since the polypeptide having the amino acid sequence shown in SEQ ID NO:1 has a high homology (about 78%) with mouse T2BP, and since it has a characteristic motif such as forkhead-associated (FHA) domain, it is apparent that this polypeptide is human T2BP. Thus, the preferred example of the polypeptide (T2BP) according to the present invention has the amino acid sequence shown in SEQ ID NO:1 or SEQ ID NO:3.

As will be described concretely in Example below, a deletion mutant consisting of the region from the 1st to 162nd amino acid of T2BP exhibited binding ability to TRAF2 (hereinafter, the 1st amino acid residue, for example, may be referred to as “1aa” for convenience, and the region from the 1st to the 162nd amino acid, for example, may be referred to as “1-162aa”). Therefore, as long as the polypeptide contains this region, it has the binding ability to TRAF2. It is also well-known in this field that, in general, the physiological activity of a polypeptide may be retained even if a small number of amino acids are substituted, deleted and/or inserted. In fact, in 1-162aa region of human and mouse T2BPs, 33 amino acid residues are different (homology of about 80%). Therefore, the polypeptide according to the present invention is defined as a polypeptide comprising a region having the same amino acid sequence as the amino acid sequence from 1st to 162nd amino acid in the amino acid sequence shown in SEQ ID NO:1 or 3 except that 1 to amino acid residue(s) is(are) substituted, deleted and/or inserted, which polypeptide has a binding ability to TRAF2. The number of amino acids which are substituted, deleted and/or inserted is preferably not more than the above-mentioned 33.

In 1-162aa of the amino acid sequence shown in SEQ ID NO:1, the amino acid residues which are different from those in the amino acid sequence of 1-162aa in the sequence shown in SEQ ID NO:3 are 2, 3, 20, 26, 28, 32, 35, 37, 38, 41, 44, 55, 57, 68, 71, 74, 77, 95, 97, 100, 101, 114, 117, 126, 127, 134, 136, 143, 145, 147, 156, 157 and 158aa. Therefore, these amino acid residues are not important and may be substituted and/or deleted, and/or 1 to 3 amino acid residues may be inserted into the sites next to these amino acid residues. Further, since the 1aa methionine is the transcription initiation codon, the 1aa of almost all proteins immediately after translation is methionine. However, since 1aa is an amino acid other than methionine in many of physiologically active proteins, the binding ability to TRAF2 is thought to be retained even without the 1aa methionine.

Similarly, in 1-162aa of the amino acid sequence shown in SEQ ID NO:3, the amino acid residues which are different from those in the amino acid sequence of 1-62aa in the sequence shown in SEQ ID NO:1 are 2, 3, 20, 27, 31, 34, 36, 37, 38, 41, 44, 55, 57, 68, 71, 74, 77, 95, 97, 100, 101, 114, 117, 126, 127, 134, 136, 143, 145, 147, 156, 157 and 158aa. Therefore, these amino acid residues are not important and may be substituted and/or deleted, and/or 1 to 3 amino acid residues may be inserted into the sites next to these amino acid residues. Further, in SEQ ID NO:1, an amino acid residue is inserted between 25aa and 26aa of SEQ ID NO:3, so that 1 to 3 amino acid residues may be inserted between 25aa and 26aa in SEQ ID NO:3. Further, since the 1aa methionine is the transcription initiation codon, the 1aa of almost all proteins immediately after translation is methionine. However, since 1aa is an amino acid other than methionine in many of physiological active proteins, the binding ability to TRAF2 is thought to be retained even without the 1aa methionine.

As mentioned above, although it was experimentally confirmed that the 1-162aa fragment has a binding ability to TRAF2, the polypeptide preferably has 1-184aa (full length) because the binding ability to TRAF2 is high. In the amino acid sequence shown in SEQ ID NO:1, the amino acid residues which are different from those in SEQ ID NO:3 are 2, 3, 20, 26, 28, 32, 35, 37, 38, 41, 44, 55, 57, 68, 71, 74, 77, 95, 97, 100, 101, 114, 117, 126, 127, 134, 136, 143, 145, 147, 156, 157, 158, 163, 165, 168, 169, 170, 171, 172, 173, 177 and 184aa. Therefore, these amino acid residues are not important and may be substituted and/or deleted, and/or 1 to 3 amino acid residues may be inserted into the sites next to these amino acid residues. Further, since the 1aa methionine is the transcription initiation codon, the 1aa of almost all proteins immediately after translation is methionine. However, since 1aa is an amino acid other than methionine in many of physiological active proteins, the binding ability to TRAF2 is thought to be retained even without the 1aa methionine. Similarly, in the amino acid sequence shown in SEQ ID NO:3, the amino acid residues which are different from those in SEQ ID NO: 1 are 2, 3, 20, 27, 31, 34, 36, 37, 38, 41, 44, 55, 57, 68, 71, 74, 77, 95, 97, 100, 101, 114, 117, 126, 127, 134, 136, 143, 145, 147, 156, 157, 158, 163, 165, 168, 169, 170, 171, 172, 173, 177 and 184aa. Therefore, these amino acid residues are not important and may be substituted and/or deleted, and/or 1 to 3 amino acid residues may be inserted into the sites next to these amino acid residues. Further, in SEQ ID NO:1, an amino acid residue is inserted between 25aa and 26aa of SEQ ID NO:3, so that 1 to 3 amino acid residues may be inserted between 25aa and 26aa in SEQ ID NO:3. Further, since the 1aa methionine is the transcription initiation codon, the 1aa of almost all proteins immediately after translation is methionine. However, since 1aa is an amino acid other than methionine in many of physiological active proteins, the binding ability to TRAF2 is thought to be retained even without the 1aa methionine.

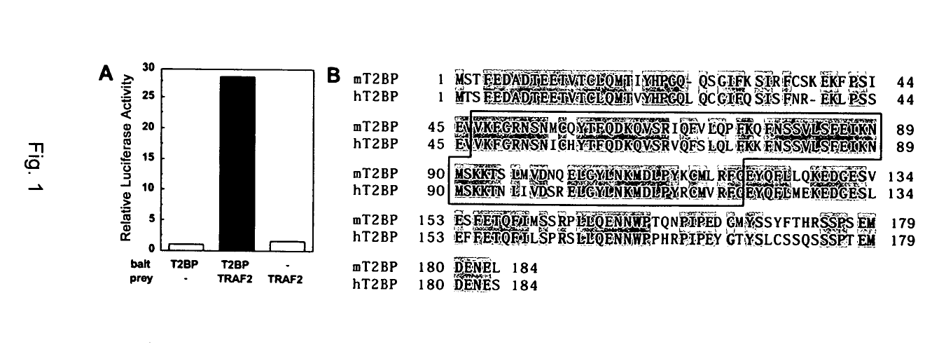

The polypeptide according to the present invention may be defined, in terms of homology, as the polypeptide comprising a region having the amino acid sequence from 1st to 162nd amino acid in the amino acid sequence shown in SEQ ID NO:1 or SEQ ID NO:3 in SEQUENCE LISTING or a region having a homology of not less than 70% with said amino acid sequence of said region, which polypeptide has a binding ability to TRAF2. Since the homology between the 1-162aa regions in SEQ ID NO:1 and SEQ ID NO:3 is about 80%, the homology of the 1-162aa is preferably not less than 80%. As mentioned above, the polypeptide preferably has the full length of the amino acid sequence shown in SEQ ID NO:1 or 3. Since the homology between the full length of SEQ ID NOs:1 and 3 is about 78%, the homology of the full length sequence is preferably not less than 78%. The “homology” used herein may be calculated by aligning the two polypeptides such that the number of matched amino acid residues is the maximum as shown in FIG. 1, B, and dividing the number of matched amino acid residues by the number of amino acid residues of the full length sequence. Such a calculation of the homology may be easily attained by a commercially available software such as BLAST. In cases where the two sequences have different lengths, the number of matched amino acid residues is divided by the number of the amino acid residues of the shorter sequence.

The polypeptide according to the present invention may be produced by the method detailed in Example below. Alternatively, since the nucleotide sequence of the cDNA coding for the polypeptide was revealed by the present invention, the polypeptide may easily be produced by preparing the nucleic acid coding for the polypeptide by a conventional method such as RT-PCR, and by expressing the nucleic acid in cells by a conventional method.

The present invention also provides nucleic acids encoding the above-described polypeptides according to the present invention. The term “nucleic acid” includes DNA and RNA. These nucleic acids may be used as templates in the production of the above-described polypeptides according to the present invention by a genetic engineering method. Specific nucleotide sequences of the preferred examples of the nucleic acid are shown in SEQ ID NOs:2 and 4 as mentioned above. The nucleic acids coding for the polypeptides having the substitution, deletion and/or insertion, which have the binding ability to TRAF2, are also the nucleic acids according to the present invention. Such a nucleic acid may preferably be the nucleic acid having the nucleotide sequence shown in SEQ ID NO:2 or 4, or one which hybridizes with the nucleic acid under stringent conditions (i.e., hybridization is performed at 50° C. to 65° C., preferably in two steps at 50° C. and 60° C., or in four steps at 50° C., 55° C., 60° C. and 65° C., using a common hybridization solution such as 5× Denhardt's reagent, 6×SSC, 0.5% SDS or 0.1% SDS).

The present invention also provides an expression vector containing the nucleic acid according to the present invention, which can express the nucleic acid in a host cell. Such an expression vector may easily be prepared by inserting the above-described nucleic acid according to the present invention into a cloning site of a commercially available expression vector, and a preparation method is described concretely in Example below. The present invention also provides a cell into which the nucleic acid according to the present invention is introduced, which expresses the polypeptide according to the present invention. Such a cell may easily be prepared by transfecting the above-described expression vector according to the present invention to a host cell by a conventional method, and a preparation method is described concretely in Example below.

The present invention also provides a nucleic acid (hereinafter also referred to as “nucleic acid for detection” for convenience) which hybridizes with the nucleic acid according to the present invention, which can be used for detection of the nucleic acid according to the present invention. Such a nucleic acid may be a primer for a nucleic acid-amplification method such as PCR or NASBA, or may be a labeled probe. In case of a primer, since amplification of the template nucleic acid does not occur unless it hybridizes with the template, it can be determined whether the nucleic acid according to the present invention which hybridizes with the primer is contained in a test sample or not based on whether the amplification occurs or not. In case of a probe, since the label of the probe is not detected unless the probe hybridizes with the nucleic acid, whether the nucleic acid of the present invention exists or not in a test sample may be determined by detecting the label of the probe. To increase the specificity of the detection, the nucleic acid for detection preferably has not less than 15 bases. In case of a primer, the number of bases is more preferably from 20 to 50, and in case of a probe, the number of bases is preferably from 20 to the full length. The nucleic acid for detection may also be used for quantification of the nucleic acid according to the present invention by using it as a primer for realtime detection PCR or by quantifying the label of the probe.

The nucleic acid-amplification methods such as PCR are well-known in this field, and the reagent kits and apparatuses are also commercially available, so that they may be easily carried out. That is, for example, a test nucleic acid serving as a template (e.g., the cDNA of the gene of the polypeptide of the present invention) and a pair of nucleic acids for detection (primers) according to the present invention are mixed in a buffer in the presence of Taq polymerase and dNTPs, and the steps of denaturation, annealing and extension are carried out by changing the temperature of the reaction mixture. Usually, the denaturation step is carried out at 90 to 95° C., the annealing step is carried out at Tm between the template and the primers or a vicinity thereof (preferably within ±4° C.), and the extension step is carried out at 72° C. which is the optimum temperature of Taq polymerase. The reaction time of each step is selected from about 30 seconds to 2 minutes. By repeating this thermal cycle for about 25 to 40 times, the region between the pair of primers is amplified. The nucleic acid-amplification method is not restricted to PCR, but other nucleic acid-amplification methods well known in the art may also be employed. By carrying out the nucleic acid-amplification method using a pair of the above-described nucleic acids for detection according to the present invention as primers and using the test nucleic acid as a template, the test nucleic acid is amplified. In contrast, in cases where the test nucleic acid is not contained in the sample, amplification does not occur. Therefore, by detecting the amplification product, whether the test nucleic acid exists in the sample or not may be determined. Detection of the amplification product may be carried out by a method in which the reaction solution after the amplification is subjected to electrophoresis, and the bands are stained with ethidium bromide or the like, or by a method in which the amplification product after electrophoresis is immobilized on a solid phase such as a nylon membrane, a labeled probe which specifically hybridizes with the test nucleic acid is hybridized with the test nucleic acid, and the label after washing is detected. Alternatively, the test nucleic acid in the sample may be quantified by the so-called realtime PCR detection using a quencher fluorescent pigment and a reporter fluorescent pigment. Since the kits for realtime PCR detection are also commercially available, realtime detection PCR may also be carried out easily. The test nucleic acid may also be semi-quantified based on the intensity of the band resulted in electrophoresis. The test nucleic acid may be a mRNA or a cDNA reverse-transcribed from a mRNA. In cases where a mRNA is amplified as the test nucleic acid, NASBA method (3SR method, TMA method) using the above-described pair of primers may also be employed. NASBA method per se is well known, and kits therefor are commercially available, so that NASBA method may easily be carried out using the above-described pair of primers.

As the probe, labeled probe obtained by labeling the above-described nucleic acid for detection with a fluorescent label, radioactive label, biotin label or the like may be used. The methods per se for labeling a nucleic acid are well known. Whether the test nucleic acid exists in the sample or not may be determined by immobilizing the test nucleic acid or amplification product thereof, hybridizing the labeled probe therewith, and measuring the label bound to the solid phase after washing. Alternatively, the nucleic acid for detection is immobilized, the test nucleic acid is hybridized therewith, and the test nucleic acid bound to the solid phase is detected by a labeled probe or the like. In such a case, the nucleic acid for detection immobilized on the solid phase is also called a probe. The methods for measuring a test nucleic acid using a nucleic acid probe are also well-known in the art, and may be attained by making contact between the nucleic acid probe and the test sample in a buffer at Tm or a vicinity thereof (preferably within ±4° C.) so as to hybridize them, and then measuring the hybridized labeled probe or the test nucleic acid bound to the immobilized probe. Such a method includes well-known methods such as Northern blot and in situ hybridization described in Example below, as well as Southern blot.

As will be concretely described in Example below, since the polypeptide of the present invention activates NF-κβ signal, and since it is a signaling pathway mediated by TNF, the NF-κβ signal may be attenuated by inhibiting the function of the polypeptide. Therefore, the polypeptide is useful as a target for the development of therapeutic drugs against inflammation, rheumatism or the like. Further, since NF-κβ signal is involved in the activation of osteoclasts, they are useful as a target for the development of therapeutic drugs against osteoporosis. Still further, since the nucleic acid for detection according to the present invention may be used for the measurement of the expression amount of T2BP gene, it may be used for monitoring the diseased state of inflammation, rheumatism, osteoporosis or the like. Still further, in cases where the therapeutic drug against inflammation, rheumatism, osteoporosis or the like is one which suppresses expression of the T2BP gene or in cases where the therapeutic drug is an antisense RNA, the nucleic acid for detection may be used for the evaluation of the pharmacological effects of these therapeutic drugs.

### EXAMPLE

The present invention will now be described more concretely by way of an Example. It should be noted that the present invention is not restricted to the Example below. All experiments were carried out by using mouse T2BP.

Materials and Methods

Cell Culture

Human embryonic kidney cell line 293 (Graham, F. L., Smiley, J., Russell, W. C., Naim, R.: J Gen Virol, 36, 59-74 (1977), obtained from the RIKEN cell bank) was cultured in Minimum Essential Medium supplemented with 10% heat-inactivated fetal bovine serum (FBS), 200 U/ml penicillin and 200 μg/ml streptomycin. 293T cells (DuBridge R B, Tang P, Hsia H C, Leong P M, Miller J H, Calos M P: Mol Cell Biol 7, 379-87 (1987)) and CHO-K1 cells (Kao, F. T., Puck, T. T.: Proc Nat Acad Sci USA, 60, 1275-1281 (1968), obtained from the RIKEN cell bank) were maintained, respectively, in Dulbecco's modified Eagle's medium and F-12 Nutrient Mixture (Ham's f-12), supplemented respectively with 10% FBS and the antibiotics.

Mammalian Two-Hybrid Assay

The assay was performed by using known method (15), that is, at first the assay samples were prepared by two-step PCR as described below using T2BP and TRAF2 cDNAs (contained in a mouse thymus-derived cDNA library from 3-day old mouse and in adult mouse testis-derived cDNA library, respectively) as templates, regions comprising their protein coding regions were amplified by PCR (first step) using the tagged gene-specific primers (forward primer T2BP, gaaggagccgccaccatgtccacctttgaagacg; forward primer TRAF2, gaaggagccgccaccatggctgcagccagtgt; designed by using known software for protein region prediction) and the primer for the vector sequence (reverse primer P8; agcggataacaatttcacacaggaaa). DNA fragments for SV40 poly-A signal and for human cytomegalo virus (CMV) immediate early promoter followed by Gal4 DNA-binding domain or herpes virus VP16 transcriptional activation domain were also amplified (sequences of forward and reverse primers and templates used herein were respectively as follows; DNA fragment of SV40 poly-A signal, gtttcctgtgtgaaattgttatccgctgcagacatgataagatacattg (forward), agcaagttcagcctggttaagatccttatcgattttaccac (reverse), pG5luc (Promega); Gal4 fragment, ccaatatgaccgccatgttggc (forward), catggtggcggctccttccggcgatacagtcaactg (reverse), pBIND (Promega); VP16 fragment, ccaatatgaccgccatgttggc (forward), catggtggcggctccttcaagtcgacggatccctggc (reverse), pACT (Promega)). In the second PCR, the first PCR product, the SV40 poly-A signal fragment and the Gal4- or VP16-fragment were ligated so as to design that the PCR product is expressed as a fusion protein with the Gal4- or VP16-domain. The sequences of the primers used in the second PCR were, gccatgttggcattgattattgac (forward) and agcaagttcagcctggttaag (reverse). The PCR products (0.13 μl) were transfected together with 20 ng of reporter plasmid pG5luc into 2.2×10⁴ of CHO-K1 cells using the transfection reagent LF2000 (Invitrogen). After 20 h of incubation, the luciferase reporter activity was measured with the Steady-Glo (trade name) Luciferase Assay System (Promega).

Co-Immunoprecipitation Analysis

The regions comprising the protein coding regions of T2BP and TRAF2 cDNAs were amplified by PCR using the gene specific primers (forward primer sequence for T2BP; gacgcgtcgaccatgtccacctttgaagacg, forward primer sequence for TRAF2; gacgcgtcgaccatggctgcagccagtgt. Reverse primer was designed for vector region, the sequence was, ccggttaagcggccgcagcggataacaatttcacacaggaaac.) and then sub-cloned into the expression vectors pCMV-HA and pCMV-Myc (both produced by Clontech), respectively. 293T cells (1×10⁶) were transfected with 2.5 μg of the expression vectors for HA-T2BP and Myc-T2BP using the a transfection reagent LF2000 (trade name). HA and Myc denote the names of the tag sequences recognized by antibodies. After 24 h of incubation, cells were harvested and lysed by TNE buffer consisting of 10 mM of Tris-HCl (pH 7.8), 1% NP40 (trade name), 0.15 M NaCl, 1 mM EDTA, 1 mM PMSF and 10 μg/ml leupeptin (PEPTIDE INSTITUTE Inc.). After centrifugation at 10,000×g for 15 min, the supernatants were isolated and immunoprecipitated with 5 μg anti-HA antibody (Santa Cruz). Detection of the co-precipitated Myc-TRAF2 was performed by Western blot analysis. Samples in Laemmuli sample buffer were boiled for 5 min and subjected to 12.5% SDS-PAGE, and proteins were transferred onto a Hybond-ECL membrane (Amersham). Western blotting was performed according to standard procedures by incubating with an anti-Myc antibody (Clontech) for 1 h and HRP-conjugated anti-mouse IgG (Amersham) for 1 h followed by washing steps. Detection of the signal was performed using the ECL system (Amersham) and X-ray film (Kodak). The supernatants described above were also subjected to direct Western blot analysis to confirm the expression of HA-T2BP and Myc-TRAF2 using the primary and secondary antibodies shown above.

Northern Analysis

T2BP and TRAF2 cDNAs were used as probes for Northern blot analysis. Probes were labeled with [³²P]dCTP using the Random Primer Labeling Kit Ver. 2 (Takara). Mouse MTN blot membrane and mouse cell line MTN blot membrane were purchased (Clontech). Hybridization was performed 30 min at 68° C. using ExpressHyb (trade name) hybridization solution (Clontech). Hybridization signals were detected by X-ray film.

Signal Transduction Pathway Analysis

The expression vectors for T2BP was co-transfected with 100 ng of the reporter vector pNFκB-Luc or pAP1-Luc (Clontech) into 5×10⁴ of 293 cells in 96-well assay plates using the transfection reagent LF2000. After 24 h of incubation, cells were treated (+) or untreated (−) by 5 ng/ml TNF for 6 h. The luciferase activity of the reporter gene was measured as described above.

Results

Identification of T2BP, a Novel TRAF2 Binding Protein with a FHA Domain

The present inventors have already reported the development of a PCR-mediated rapid sample preparation and a high-throughput assay system based on the mammalian two-hybrid method (15). Using this system, approximately 6,000 cDNAs, which were derived from mouse full-length enriched libraries, were assayed by the matrix approach. A novel TRAF2 interacting protein, designated as T2BP (TRAF2 binding protein), was identified when TRAF2 fused to the VP16 transcriptional activation domain was used as a prey (FIG. 1A). The cDNA sequence of mouse T2BP (SEQ ID NO:4) contains five A+T-rich ATTTA motif in the 3′-UTR. This was found in many short-lived mRNAs such as those of cytokines and proto-oncogenes and is therefore a potent destabilizing element (16, 17). As shown in FIG. 1B, T2BP consists of 184 amino acid residues and is calculated with an isoelectric point (pI) of 4.79 and a molecular weight of 21,560. The motif analysis by Pfam motif database search (http://pfam.wustl.edu/index.html) exhibited a forkhead-associated (FHA) domain in the central region of T2BP that is known as a phosphopeptide binding motif (18, 19). Further, homology searches by BLAST identified the human orthologue of mouse T2BP (FIG. 1B and SEQ ID NO:1 and NO:2). The mouse and human T2BP are highly conserved throughout the whole amino acid sequences.

FIG. 1A shows the result of mammalian two-hybrid assay. The relative luciferase activity of the reporter gene was measured when prey-TRAF2 and/or bait-T2BP were co-transfected with the reporter vector pG5luc into CHO-K1 cells. Relative values were calculated by using mean values for either prey-TRAF2 or bait-T2BP transfection as a base.

FIG. 1B shows amino acid sequences of mouse and human T2BPs (mT2BP and hT2BP, respectively). Identical amino acid residues are shaded. The FHA domain is indicated by a box. The amino acid sequences of m- and hT2BP were deduced from the cDNAs.

T2BP Interacts with the TRAF Domain of TRAF2

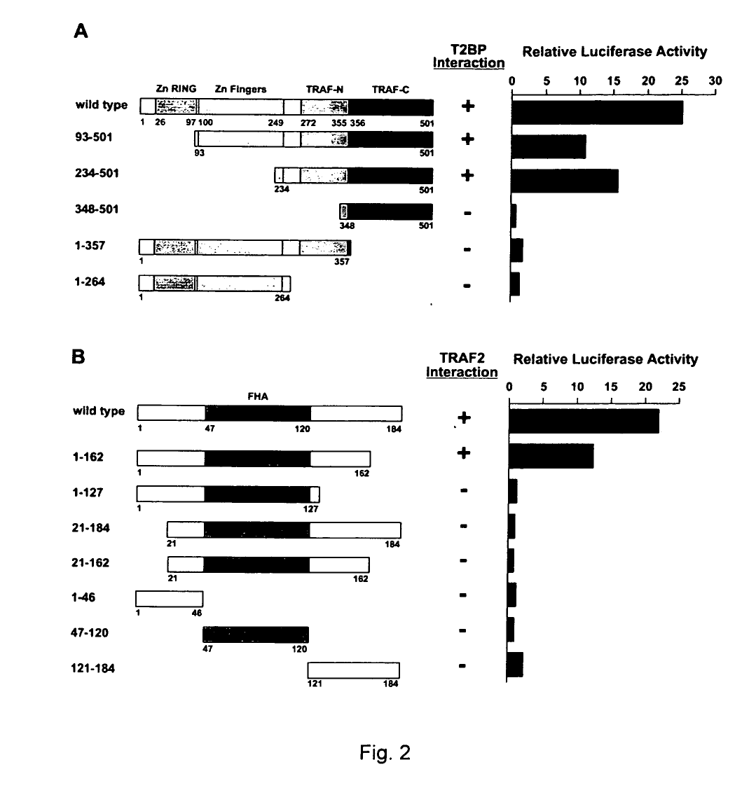

TRAF2 is an adaptor protein with several well-known motifs (1-4), as shown in FIG. 2A. To determine which motif is responsible for the interaction with T2BP, the present inventors examined interaction of T2BP with wild-type TRAF2 or the deletion mutants using their mammalian two-hybrid method. The result showed that the minimal interacting region in TRAF2 includes the carboxyl-terminal half, which is known as the TRAF domain. The TRAF domain can be divided into TRAF-N and TRAF-C subdomains (20). Since T2BP did neither interact with TRAF2[1-357] nor with TRAF2[348-501], both subdomains are essential for the interaction with T2BP. The same result was also obtained using in vitro GST-pull down assays. Next, the inventors examined the region in T2BP responsible for TRAF2 interaction (FIG. 2B). All of the examined T2BP deletion mutants, except for T2BP[1-162], lost the ability to bind to TRAF2.

FIG. 2A shows schematic representation of TRAF2 and the deletion mutants and their interaction with wild-type T2BP. FIG. 2B shows schematic representation of T2BP and the deletion mutants and their interaction with wild-type TRAF2. Co-immunoprecipitation analysis of the interaction between T2BP and TRAF2

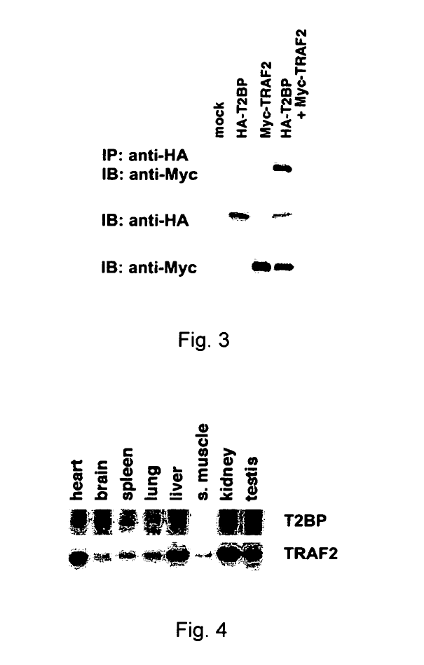

The interaction between T2BP and TRAF2 was confirmed in vivo by co-immunoprecipitation (FIG. 3). The present inventors constructed expression vectors for HA-tagged T2BP and Myc-tagged TRAF2. Those expression vectors were transfected into 293T cells and the cell extracts were subjected to the immunoprecipitation using an anti-HA antibody. Western blot analysis using an anti-Myc antibody (FIG. 3, top) showed that Myc-TRAF2 was specifically co-immunoprecipitated with HA-T2BP.

The middle of FIG. 3 shows the result of the Western blot analysis to which the extracts above were subjected, confirming the expression of HA-T2BP, and the bottom of FIG. 3 shows the result confirming the expression of Myc-TRAF2 in a similar manner. In FIG. 3, IP and IB denote immunoprecipitation and immunoblotting, respectively.

Expression Profiles of T2BP

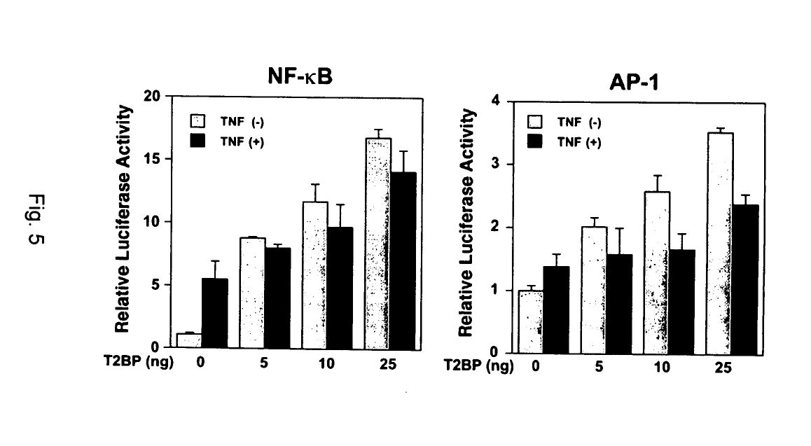

Since co-expression and co-localization of TRAF2 and T2BP are essential for their interactive functions, the expression profiles of T2BP and TRAF2 were examined by Northern blot analysis. When T2BP cDNA was used as a probe, a 2.3 kb signal was detected, which was reasonable size corresponding to the obtained cDNA size of 2.0 kb. T2BP was ubiquitously expressed in adult major tissues (FIG. 4). In addition to the major signal, two weaker signals of 3.0 and 4.0 kb for T2BP were also observed. As the result of using TRAF2 cDNA as a probe indicated, TRAF2 gene was similarly expressed in all adult major tissues, which was consistent with the previous report (21). Next, various kinds of mouse cultured cell lines were examined to determine which cell line was expressing T2BP at a high level. The cell lines below were examined: PU5-1.8(PU5-R), RAW264.7, K-BALB(K-234), M-MSV-BALB/3T3, L-M, P19, Hepal-6, R1.1, L1210, P388D1, P815 and NB41A3. Among these, the cell lines derived from immune system cells such as RAW264.7, L1210 and P388D1 were found to express T2BP at a high level.

Activation of NF-kB and AP-1 in T2BP-Transfected 293 Cells

TNF-induced activation of NF-κB and AP-1 is well established in several cell lines including 293 cells, where TRAF2 is known to play a key role for the signal transduction (11, 22). To evaluate the effect of T2BP to these pathways, T2BP was overexpressed in 293 cells together with a reporter vector that allowed to detect the activation of NF-κB by luciferase activity (FIG. 5, left). When 293 cells that were transfected with only the reporter vector were treated with TNF, an activation of NF-κB was clearly detected. Overexpression of T2BP activated NF-κB without TNF treatment in a dose dependent manner. TNF treatment of T2BP-transfected 293 cells showed activation of NF-κB similar or slightly less than those of the TNF-untreated T2BP-transfected cells. The same type of experiments was performed to evaluate the effect of T2BP for the activation of AP-1 (FIG. 5, right). Activation of AP-1 in TNF-treated control 293 cells was detected. Overexpression of T2BP in TNF-untreated 293 cells induced AP-1 activation in a dose-dependent manner. TNF treatment of T2BP-transfected cells showed less AP-1 activation compared with that of TNF-untreated T2BP-transfected cells. Thus, the results showed that T2BP overexpression activated both NF-κB and AP-1 without TNF treatment.

### REFERENCES

1. Bradley, J. R., and Pober, J. S. (2001) Oncogene 20, 6482-6491.
2. Wajant, H., Henkler, F., and Scheurich, P. (2001) Cell Signal 13, 389-400.
3. Inoue, J., Ishida, T., Tsukamoto, N., Kobayashi, N., Naito, A., Azuma, S., and Yamamoto, T. (2000) Exp Cell Res 254, 14-24.
4. Wajant, H., Grell, M., and Scheurich, P. (1999) Cytokine Growth Factor Rev 10, 15-26.
5. Baker, S. J., and Reddy, E. P. (1998) Oncogene 17, 3261-3270.
6. Hsu, H., Xiong, J., and Goeddel, D. V. (1995) Cell 81, 495-504.
7. Shu, H. B., Takeuchi, M., and Goeddel, D. V. (1996) Proc Natl Acad Sci U S A93, 13973-13978.
8. Stanger, B. Z., Leder, P., Lee, T. H., Kim, E., and Seed, B. (1995) Cell 81, 513-523.
9. McCarthy, J. V., Ni, J., and Dixit, V. M. (1998) J Biol Chem 273, 16968-16975.
10. Nishitoh, H., Saitoh, M., Mochida, Y., Takeda, K., Nakano, H., Rothe, M., Miyazono, K., and Ichijo, H. (1998) Mol Cell 2, 389-395.
11. Baud, V., and Karin, M. (2001) Trends Cell Biol 11, 372-377.
12. Baud, V., Liu, Z. G., Bennett, B., Suzuki, N., Xia, Y., and Karin, M. (1999) Genes Dev 13, 1297-1308.
13. Chinnaiyan, A. M., O'Rourke, K., Tewari, M., and Dixit, V. M. (1995) Cell 81, 505-512.
14. Hu, W. H., Johnson, H., and Shu, H. B. (2000) J Biol Chem 275, 10838-10844.
15. Suzuki, H., Fukunishi, Y., Kagawa, I., Saito, R., Oda, H., Endo, T., Kondo, S., Bono, H., Okazaki, Y., and Hayashizaki, Y. (2001) Genome Res 11, 1758-1765.
16. Wilson, G. M., and Brewer, G. (1999) Prog Nucleic Acid Res Mol Biol 62, 257-291.
17. Guhaniyogi, J., and Brewer, G. (2001) Gene 265, 11-23.
18. Li, J., Lee, G. I., Van Doren, S. R., and Walker, J. C. (2000) J Cell Sci 113 Pt 23, 4143-4149.
19. Durocher, D., Henckel, J., Fersht, A. R., and Jackson, S. P. (1999) Mol Cell 4, 387-394.
20. Cheng, G., Cleary, A. M., Ye, Z. S., Hong, D. I., Lederman, S., and Baltimore, D. (1995) Science 267, 1494-1498.
21. Rothe, M., Wong, S. C., Henzel, W. J., and Goeddel, D. V. (1994) Cell 78, 681-692.
22. Rothe, M., Sarma, V., Dixit, V. M., and Goeddel, D. V. (1995) Science 269, 1424-1427.
23. Hofmann, K., and Bucher, P. (1995) Trends Biochem Sci 20, 347-349.
24. Arch, R. H., Gedrich, R. W., and Thompson, C. B. (2000) Biochem Biophys Res Commun 272, 936-945.
25. Takeuchi, M., Rothe, M., and Goeddel, D. V. (1996) J Biol Chem 271, 19935-19942.
26. Qian, Y., Commane, M., Ninomiya-Tsuji, J., Matsumoto, K., and Li, X. (2001) J Biol Chem 276, 41661-41667.
27. Rothe, J., Gehr, G., Loetscher, H., and Lesslauer, W. (1992) Immunol Res 11, 81-90.
28. Wajant, H., and Scheurich, P. (2001) Int J Biochem Cell Biol 33, 19-32.
29. Gravallese, E. M., Galson, D. L., Goldring, S. R., and Auron, P. E. (2001) Arthritis Res 3, 6-12.
30. Kobayashi, N., Kadono, Y., Naito, A., Matsumoto, K., Yamamoto, T., Tanaka, S., and Inoue, J. (2001) Embo J 20, 1271-1280.
31. Zhang, Y. H., Heulsmann, A., Tondravi, M. M., Mukherjee, A., and Abu-Amer, Y. (2001) J Biol Chem 276, 563-568.

1. A polypeptide comprising a region having the amino acid sequence from 1st to 162nd amino acid in the amino acid sequence shown in SEQ ID NO:1 in SEQUENCE LISTING or a region having the same amino acid sequence as said amino acid sequence from 1st to 162nd amino acid in the amino acid sequence shown in SEQ ID NO:1 except that 1 to 40 amino acid residue(s) is(are) substituted, deleted and/or inserted, which polypeptide has a binding ability to TRAF2.

2. The polypeptide according to claim 1, comprising said region having the amino acid sequence from 1st to 162nd amino acid in the amino acid sequence shown in SEQ ID NO:1 in SEQUENCE LISTING or a region having the same amino acid sequence as said amino acid sequence from 1st to 162nd amino acid in the amino acid sequence shown in SEQ ID NO:1 except that 1 to 33 amino acid residue(s) is(are) substituted, deleted and/or inserted.

3. The polypeptide according to claim 2, comprising a region having the same amino acid sequence as said amino acid sequence from 1st to 162nd amino acid in the amino acid sequence shown in SEQ ID NO:1 except that at least one amino acid selected from the group consisting of 1st, 2nd, 3rd, 20th, 26th, 28th, 32nd, 35th, 37th, 38th, 41st, 44th, 55th, 57th, 68th, 71st, 74th, 77th, 95th, 97th, 100th, 101st, 114th, 117th, 126th, 127th, 134th, 136th, 143rd, 145th, 147th, 156th, 157th and 158th amino acid(s) is(are) substituted and/or deleted, and/or 1 to 3 amino acid(s) each is(are) inserted into site(s) next to said at least one amino acid selected from said group.

4. The polypeptide according to claim 2, comprising said region having the amino acid sequence from 1st to 162nd amino acid in the amino acid sequence shown in SEQ ID NO:1 in SEQUENCE LISTING.

5. The polypeptide according to claim 2, having the same amino acid sequence as shown in SEQ ID NO:1 in SEQUENCE LISTING, or having the same amino acid sequence as shown in SEQ ID NO:1 except that at least one amino acid selected from the group consisting of 1st, 2nd, 3rd, 20th, 26th, 28th, 32nd, 35th, 37th, 38th, 41st, 44th, 55th, 57th, 68th, 71st, 74th, 77th, 95th, 97th, 100th, 101st, 114th, 117th, 126th, 127th, 134th, 136th, 143rd, 145th, 147th, 156th, 157th, 158th, 163rd, 165th, 168th, 169th, 170th, 171st, 172nd, 173rd, 177th and 184th amino acids is substituted and/or deleted, and/or 1 to 3 amino acid(s) each is(are) inserted into site(s) next to said at least one amino acid selected from said group, which polypeptide has a binding ability to TRAF2.

6. The polypeptide according to claim 5, having the amino acid sequence shown in SEQ ID NO:1 in SEQUENCE LISTING.

7. A polypeptide comprising a region having the amino acid sequence from 1st to 162nd amino acid in the amino acid sequence shown in SEQ ID NO:3 in SEQUENCE LISTING or a region having the same amino acid sequence as said amino acid sequence from 1st to 162nd amino acid in the amino acid sequence shown in SEQ ID NO:3 except that 1 to 40 amino acid residue(s) is(are) substituted, deleted and/or inserted, which polypeptide has a binding ability to TRAF2.

8. The polypeptide according to claim 7, comprising said region having the amino acid sequence from 1st to 162nd amino acid in the amino acid sequence shown in SEQ ID NO:3 in SEQUENCE LISTING or a region having the same amino acid sequence as said amino acid sequence from 1st to 162nd amino acid in the amino acid sequence shown in SEQ ID NO:3 except that 1 to 33 amino acid residues is(are) substituted, deleted and/or inserted.

9. The polypeptide according to claim 8, comprising a region having the same amino acid sequence as said amino acid sequence from 1st to 162nd amino acid in the amino acid sequence shown in SEQ ID NO:3 except that at least one amino acid selected from the group consisting of 1st, 2nd, 3rd, 20th, 27th, 31st, 34th, 36th, 37th, 38th, 41st, 44th, 55th, 57th, 68th, 71st, 74th, 77th, 95th, 97th, 100th, 101st, 114th, 117th, 126th, 127th, 134th, 136th, 143rd, 145th, 147th, 156th, 157th and 158th amino acid(s) is(are) substituted and/or deleted, and/or 1 to 3 amino acid(s) each is(are) inserted into site(s) next to said at least one amino acid selected from said group.

10. The polypeptide according to claim 8, comprising said region having the amino acid sequence from 1st to 162nd amino acid in the amino acid sequence shown in SEQ ID NO:3 in SEQUENCE LISTING.

11. The polypeptide according to claim 8, having the same amino acid sequence as shown in SEQ ID NO:3 in SEQUENCE LISTING, or having the same amino acid sequence as shown in SEQ ID NO:3 except that at least one amino acid selected from the group consisting of 1st, 2nd, 3rd, 20th, 27th, 31st, 34th, 36th, 37th, 38th, 41st, 44th, 55th, 57th, 68th, 71st, 74th, 77th, 95th, 97th, 100th, 101st, 114th, 117th, 126th, 127th, 134th, 136th, 143rd, 145th, 147th, 156th, 157th, 158th, 163rd, 165th, 168th, 169th, 170th, 171st, 172nd, 173rd, 177th and 184th amino acid(s) is(are) substituted and/or deleted, and/or 1 to 3 amino acid(s) each is(are) inserted into site(s) next to said at least one amino acid selected from said group and/or into site between 25th and 26th amino acids.

12. The polypeptide according to claim 11, having the amino acid sequence shown in SEQ ID NO:3 in SEQUENCE LISTING.

13. A polypeptide comprising a region having the amino acid sequence from 1st to 162nd amino acid in the amino acid sequence shown in SEQ ID NO:1 or SEQ ID NO:3 in SEQUENCE LISTING or a region having a homology of not less than 70% with said amino acid sequence of said region, which polypeptide has a binding ability to TRAF2.

14. The polypeptide according to claim 13, wherein said homology is not less than 80%.

15. A polypeptide having an amino acid sequence with a homology of not less than 70% with the amino acid sequence shown in SEQ ID NO:1 or SEQ ID NO:3 in SEQUENCE LISTING.

16. The polypeptide according to claim 15, wherein said homology is not less than 78%.

17. A nucleic acid coding for said polypeptide according to claim 1, 7, 13 or 15.

18. The nucleic acid according to claim 17, having the nucleotide sequence shown in SEQ ID NO:2 or SEQ ID NO:4 in SEQUENCE LISTING or a nucleic acid which hybridizes with a nucleic acid complementary to said nucleic acid having the nucleotide sequence shown in SEQ ID NO:2 or SEQ ID NO:4 under stringent conditions.

19. An expression vector containing said nucleic acid according to claim 17, which can express said nucleic acid in a host cell.

20. A cell into which said nucleic acid according to claim 17 is introduced, which expresses said polypeptide.

21. A nucleic acid which hybridizes with said nucleic acid according to claim 17, which can be used for detection of said nucleic acid.

22. The nucleic acid according to claim 21, which hybridizes with said nucleic acid having the nucleotide sequence shown in SEQ ID NO:2 or SEQ ID NO:4 in SEQUENCE LISTING.

23. The nucleic acid according to claim 21, which is a primer or a probe.

24. The nucleic acid according to claim 21, comprising not less than 15 bases.

25. A method for measuring said a nucleic acid comprising contacting said probe according to claim 23 with said nucleic acid so as to hybridize them, and measuring the hybridized nucleic acid.

26. A method for measuring a nucleic acid comprising carrying out a nucleic acid-amplification method using a pair of primers according to claim 23 as primers and using said nucleic acid as a template, and measuring amplified product.
